(19) United States
(12) Patent Application Publication
(10) Pub. No.: US 2009/0216187 A1
(43) Pub. Date: Aug. 27, 2009

(54) Method of Treatment of Vascular Diseases

(76) Inventors: Walter Wohlgemuth, Augsburg (DE); Hermann Wengenmair, Bonstetten (DE); Jurgen Kopp, Aystetten (DE)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/035,775

(22) Filed: Feb. 22, 2008

(51) Int. Cl. A61F 2/84; A61M 25/10

(52) U.S. Cl. 604/103.1; 623/1.11

(57) ABSTRACT

The present invention relates in general to the field of methods for the treatment of vascular diseases, namely to a method of a minimal invasive surgery. More precisely the present invention relates to a method of a minimal invasive surgery for the treatment of a human suffering from a vascular disease comprising endovascular angioplasty and endovascular balloon brachytherapy using a liquid beta-radiation emitting source.

The present invention relates in general to the field of methods for the treatment of vascular diseases, namely to a method of a minimal invasive surgery.

### FIELD OF THE INVENTION

The peripheral arterial occlusive disease with disturbed blood flow in legs caused by stenosis or occlusions of the afferent peripheral arteries is a widespread disease. For instance, alone in Germany more than 3.3 million people suffer from such a disease.

First aim of an invasive therapy for the treatment of a stenosed artery is the native vessel recanalization which can be achieved using a method of endovascular dilatation such as percutaneous transluminal angioplasty (PTA) and/or stent implantation. The PTA increases the lumen by radial expansion which has been proven to be a successful method of treatment for opening blocked or stenosed vessel and restoring blood flow. In case such a dilatation is not possible, potentially an artificial collateral circulation (bypass surgery) has to be created. Although effective in providing an alternate route for blood flow such a bypass surgery is a high-risk and high-cost procedure. The main advantage of a PTA lies in reducing morbidity and avoiding the immediate post-operative discomforts associated with a bypass surgery.

However, beside the success of methods of endovascular minimal invasive dilatation for the treatment of peripheral arterial vasoconstrictions, to date re-narrowing (restenosis) of the expanded lumen is still an essential problem, especially in the area of femoropopliteal vessels of the leg. In an unselected patient population (all femoropopliteal angioplasties over 5 years) the cumulative 2-years patency rate of the treated arteries was only 46% [Stanley B., Teague B., Raptis S., Taylor D J, Berce M. “Efficacy of balloon angioplasty of the superficial femoral artery and popliteal artery in the relief of leg ischemia” J. Vase. Surg. 1996; 23: 679-685]. Further, the treatment of long and multifocal femoropopliteal stenosis and occlusions resulted in a 6-month patency rate of only 23.1% [Murray R. R. Jr., Hewes R. C., White R. I. Jr., Mitchell S. E., Auster M., Chang R., Kadir S., Kinnison M. L., Kaufnan S. L. “Long-segment femoropopliteal stenoses: is angioplasty a boon or a bust?” Radiology 1987; 162: 473-476] and a 3-years patency rate of 20%, respectively [Jorgensen B., Tonnesen K. H., Holstein P. “Late hemodynamic failure following percutaneous transluminal angioplasty for long and multifocal femoropopliteal stenoses” Cardiovasc. Intervent. Radiol. 1991; 14: 290-292].

The finding that restenosis results from uncontrolled proliferation that occurs after balloon injury of the arterial endothelium during angioplasty [Austin G. E., Ratliff N. B., Hollman J., Tabei S., Phillips D. F. “Intimal proliferation of smooth muscle cells as an explanation for recurrent coronary artery stenosis after percutaneous transluminal coronary angioplasty” J. Am. Coll. Cardiol. 1985; 6(2): 369-375; Liu M. W., Roubin G. S., King S. B. “3rd. Restenosis after coronary angioplasty. Potential biologic determinants and role of internal hyperplasia” Circulation. 1989; 9(6): 1374-1387] established the idea of a potential protective efficiency of a local vascular brachytherapy. The presumption of an anti-proliferative effect of different radiation emitting sources has been proved using both vascular cell cultures [Rosen E. M., Goldberg I. D., Myrick K. V., Levenson S. “Radiation survival properties of cultured vascular smooth muscle cells” Radiat. Res. 1984; 100(1): 182-191; Fischer-Dzoga K., Dimitrievich G. S., Griem M. L., Radiosensitivity of vascular tissue. II. Differential radiosensitivity of aortic cells in vitro” Radiat. Res. 1984; 99(3): 536-546] and animal experimental studies which showed a prevention of an occurrence of restenosis after PTA [Wiedermann J. G., Marboe C., Amols H., Schwartz A., Weinberger J., “Intracoronary irradiation markedly reduces neointimal proliferation after balloon angioplasty in swine: persistent benefit at 6-month follow-up” Am. Coll. Cardiol. 1995; 25(6): 1451-1456. Waksman R., Rodriguez J. C., Robinson K. A., Cipolla G. D., Crocker I. R., Scott N. A., King S. B. 3rd, Wilcox J. N. “Effect of intravascular irradiation on cell proliferation, apoptosis, and vascular remodeling after balloon overstretch injury of porcine coronary arteries” Circulation Sep. 16, 1997; 96(6):1944-52].

Brachytherapy is in particular effective and has low side effects if it is endovascularly and directly applied to the target vessel, and in addition, preferably without any irradiation of adjacent tissue. To date a multiplicity of prospective, placebo-controlled clinical studies with respect to the principal efficiency of an endovascular brachytherapy (EVBT) exist which prove unambiguously the advantage according to evidence based criteria [Wohlgemuth W A, Bohndorf K. “Endovascular brachytherapy to prevent restenosis after angioplasty” Rofo. 2003; 175(2): 246-252]. The applied and authorized systems comprise wire-based gamma (Iridium-192) or beta-(Yttrium-90/Strontium-90) radiation emitting sources.

In order to receive a homogenous dose distribution in the vessel such wire-based radiation emitting sources have to be centered within the vascular lumen using special means of catheter.

However, despite the clear assured efficiency of an endovascular brachytherapy, it has been barely entered the routine clinical use beyond its use in clinical studies [Waksman R., Weinberger J., “Coronary brachytherapy in the drug-eluting stent era: don't bury it alive” Circulation. 2003, 29; 108(4): 386-388]. This can be explained by several reasons.

One reason is the radiation exposure of the patient and the attending therapist by the application of gamma-emitters. Gamma-rays are known to completely penetrate the body. Therefore, the use of gamma-radiation emitting sources requires an extensive shield during treatment. Thus, performing a brachytherapy using standard means requires that the patient has to be brought to a radiation therapy unit in order to protect the attending staff during the treatment. However, in most cases the location of the radiation emitting source introduced into a patient's body cannot be monitored in a radiation therapy unit. This bears certain risk factors. Therefore, the patient has to be transported back to the angiography unit after the brachytherapy in order to perform a control angiography. This procedure is time-consuming and further, does increase the complexity and the risk of treatment. In addition, the patient itself can not be protected against the gamma-radiation which acts in the whole body and which might generate potential damages of adjacent tissue or adjacent organs (deterministic or stochastic radiation damages).

One technical problem related to the application of a wire-based conventional technique results from the needed homogeneity of the irradiation. Utilizing wire-based irradiation systems an additional centering catheter [Verin V., Urban P., Popowski Y., Schwager M., Nouet P., Dorsaz P. A., Chatelain P., Kurtz J. M., Rutishauser W. “Feasibility of intracoronary beta-irradiation to reduce restenosis after balloon angioplasty. A clinical pilot study” Circulation. 1997, 4; 95(5): 1138-1144; Raizner A. E., Oesterle S. N., Waksman R., Serruys P. W., Colombo A., Lim Y. L., Yeung A. C., van der Giessen W. J., Vandertie L., Chiu J. K., White L. R., Fitzgerald P. J., Kaluza G. L., Ali N. M. “Inhibition of restenosis with beta-emitting radiotherapy: Report of the Proliferation Reduction with Vascular Energy Trial (PREVENT)” Circulation. 2000, 29; 102(9): 951-958] has been proposed in order to improve the results via a more homogenous irradiation of the vessel wall. The centering catheter tempts to center the radiation-active wire using at least two additional balloons which are inflated within the vessel. However, the necessary use of such a centering catheter prolongs the intervention time and complexity. Further, the centering catheter itself can lead to vessel damages such as intimal dissection and/or peripheral embolisation. [Hoftnann W. J., Kopp M., Kofler B., Ugurluoglu A., Kogelnik D., Magometschnigg H. “Preliminary observations on the need for control angiography after peripheral endovascular brachytherapy using a centering balloon.” J. Endovase. Ther. 2002; 9(2): 241-245]. Also, the use of a centering catheter causes additional costs.

Next to the above-mentioned radioactive wires a new concept for endovascular brachytherapy is the use of a liquid-filled balloon containing a beta-emitting radioisotope. A major advantage is the optimal delivery of the radioactivity to the vessel wall. One possible radioisotope is Rhenium-188 which is a high-energy beta-emitter that is routinely available from a Wolfram-188/Rhenium-188 generator in liquid form. However, a potential problem applying a liquid-filled balloon containing Rhenium-188 is the rupture of the balloon. In order to increase the lumen by radial expansion of the stenotic region inflating the balloon catheter requires high pressures of about 10 to 15 bar. The probability of a balloon rupture at high pressures such as 10 to 15 bar is approximately 1:10.000 [Knapp F. F. Jr., Guhlke S., Beets A. L., Lin W. Y., Stabin M., Amols H., Weinberger J. “Endovascular beta irradiation for prevention of restenosis using solution radioisotopes: pharmacologic and dosimetric properties of rhenium-188 compounds.” Cardiovasc. Radiat. Med. 1999; 1(1): 86-97]. A rupture of the balloon during the treatment results in an accidental incorporation of the liquid Rhenium-188. The theoretical calculated total body dose after the complete accidental incorporation of a balloon filling of Rhenium-188-DPTA of about 3 ml is approximately 63 mGy [Lee J., Lee D. S., Kim K. M., Yeo J. S., Cheon G. J. Kim S. K., Ahn J. Y. Jeong J. M. Chung J. K., Lee M. C. “Dosimetry of rhenium-188 diethylene triamine penta-acetic acid for endovascular intra-balloon brachytherapy after coronary angioplasty.” Eur. J. Nucl. Med. 2000; 27(1): 76-82]. This value is in the range of a normal radiological intervention [Kotzerke J., Fenchel S., Guhlmann A., Stabin M., Rentschler M., Knapp F. F. Jr., Reske S. N. “Pharmacokinetics of 99Tcm-pertechnetate and 188Re-perrhenate after oral administration of perchlorate: option for subsequent care after the use of liquid 188Re in a balloon catheter.” Nucl. Med. Commun. 199; 19(8): 795-801].

To date one accidental Rhenium-188 incorporation caused by balloon rupture has been reported. The measured total body dose of the treated patient was 113 mGy (image-based analysis) and 88 mGy (biological analysis), respectively. Thus, the measured radiation exposures were within the theoretical calculated ranges. [Paeng J. C., Lee D. S., Kang W. J., Yang H. M., Chung J. K., Jeong J. M., Chae I. H., Lee M. M., Lee M. C. “Dosimetry in leakage of (188)Re-DTPA during intracoronary balloon brachytherapy” Eur. J. Nucl. Med. Mol. Imaging. 2003; 30(9): 1263-1265]. Rhenium-188 has a physical half-time of 16.9 hours and is renal eliminated with an elimination half-life of about 7 h after incorporation (animal experimental data). Therefore, Rhenium-188 will remain in the body for about 2 days after incorporation. In the body Rhenium-188 is mainly absorbed by the thyroid (0.0167 mGy/MBq) and the stomach (0.013 mGy/MBq) [Knapp F. F., Jr., Guhlke S., Beets A. L., Lin W. Y., Stabin M., Amols H., Weinberger J. “Endovascular beta irradiation for prevention of restenosis using solution radioisotopes: pharmacologic and dosimetric properties of rhenium-188 compounds.” Cardiovasc. Radiat. Med. 1999; 1(1): 86-97; Lin W. Y., Tsai S. C., Hsieh B. T. Lee T. W., Ting G., Wang S. J “Evaluation of three rhenium-188 candidates for intravascular radiation therapy with liquid-filled balloons to prevent restenosis” J. Nucl. Cardiol. 2000; 7(1): 37-42]. However, the thyroid and stomach uptake can be blocked or displaced by oral perchlorate administration [Knapp F. F. Jr., Guhlke S., Beets A. L., Lin W. Y., Stabin M., Amols H., Weinberger J. “Endovascular beta irradiation for prevention of restenosis using solution radioisotopes: pharmacologic and dosimetric properties of rhenium-188 compounds” Cardiovasc. Radiat. Med. 1999; 1(1): 86-97]. Therefore, the whole-body radiation can be reduced to 38% by subsequent oral administration of perchlorate [Kotzerke J., Fenchel S., Guhlmann A., Stabin M., Rentschler M., Knapp F. F. Jr., Reske S. N. “Pharmacokinetics of 99Tcm-pertechnetate and 188Re-perrhenate after oral administration of perchlorate: option for subsequent care after the use of liquid 188Re in a balloon catheter.” Nuel. Med. Commun. 1998; 19(8): 795-801]. Thus, Rhenium-188 has favourable properties for endovascular brachytherapy using a balloon catheter. However, the balloon rupture carries a certain risk for the patient to be treated.

Thus, in view of the above, a safer method for the treatment of vascular diseases would be desirable. It is an object of the present invention to provide a method of a minimal invasive surgery which reduces the risk of an incorporation of the radiation emitting source.

### SUMMARY OF THE INVENTION

The object of the present invention is achieved by a method of a minimal invasive surgery for the treatment of a human suffering from a vascular disease comprising the steps of a) performing an endovascular angioplasty of a stenotic region, and b) performing an endovascular brachytherapy at the stenotic region using a liquid beta-radiation emitting source. Preferably, step b) is performed after step a) has been completed.

### DESCRIPTION OF PREFERRED EMBODIMENTS OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In context with the present invention “endovascular angioplasty” means any percutaneous transluminal method of decreasing stenosis within a blood vessel, whether caused by the existence of an atheromatous plaque, thrombosis, embolus, and/or mineral deposit, by any of a number of means such as balloon dilation, thermal ablation, laser atherectomy, mechanical shaving, extraction, or ultrasonic pulverization.

The term “stenotic region” means in context with the present invention a pathologic narrowing of a blood vessel.

In context with the present invention “endovascular brachytherapy” means any percutaneous transluminal angioplasty method known by the person skilled in the art, wherein a liquid-filled container such as a catheter or balloon catheter containing a beta-emitting radioisotope is placed in juxtaposition to a stenotic region.

Performing an endovascular angioplasty of a stenotic region according to step a) opens the blocked or stenosed vessel and restores blood flow. During a subsequent endovascular brachytherapy according to step b) the stenotic region is irradiated in order to prevent at least partially a restenosis.

Thus, the present invention provides a method which reduces the risk of an incorporation of the radiation emitting source, since the radiation emitting source is used only during the endovascular brachytherapy of a stenotic region for which no high pressures are necessary and after the stenotic region already has been dilated, while conventionally, endovascular balloon brachytherapy includes a one-step simultaneous dilatation and irradiation of a stenotic region using the radiation emitting fluid.

Since the method of the present invention reduces the risk of an incorporation of the radiation emitting source and furthermore uses beta-radiation instead it should promote the entry of endovascular brachytherapy in the routine clinical supply.

In one embodiment of the method of a minimal invasive surgery according to the present invention step a) comprises the steps of (i) placing a balloon catheter in juxtaposition to the stenotic region, (ii) inflating the balloon catheter, and (iii) deflating the balloon catheter.

Preferably, a biocompatible fluid such as a sterile saline solution is used in step (ii). The term “balloon catheter” comprises any balloon catheter as known by a person skilled in the art which is suitable to perform an endovascular angioplasty and/or endovascular balloon brachytherapy according to the present invention.

In a preferred embodiment of the method of a minimal invasive surgery according to the present invention step (ii) is performed with a pressure between 8 to 17 bar, preferably between 10 to 15 bar. Step (ii) can be performed using standard techniques and common materials as known by a person skilled in the art. This step increases the vascular lumen by radial expansion and therefore restores blood flow. In addition, the applied pressure provides a certain homogenisation of the vascular plaque and the thickness of the vessel wall including that the plaque will become more uniform.

In another preferred embodiment of the method of a minimal invasive surgery according to the present invention step a) further comprises implanting a stent in juxtaposition to the stenotic region.

Stents are commonly used in angioplasty to restore and maintain adequate blood flow to the heart and to prevent the artery wall from collapsing or closing again. A stent acts as a scaffold in order to provide structural support for a vessel. A stent, or a small, expandable wire tube, is often used for the treatment of coronary artery disease.

In the context of the present invention “stent” means a slender thread, rod, or catheter lying within the lumen of a vessel in order to provide support and to assure patency of an intact but contracted lumen. A stent can also include more than one stent and can also include an assembly, where a graft material is sandwiched between two stents.

During endovascular angioplasty, the balloon catheter is placed inside the stent and inflated which opens the stent and pushes it into place against the artery wall. The stent is left permanently and often, because the stent is mesh-like, the cells lining the blood vessel grow through and around the stent to help secure it.

In a further preferred embodiment of the method of a minimal invasive surgery according to the present invention step a) further comprises withdrawing the balloon catheter. Preferably, withdrawing only occurs if a different catheter is used in step b).

In one embodiment of the method of a minimal invasive surgery according to the present invention step b) comprises the steps of (J) injecting the liquid beta-radiation emitting source into a catheter, (II) leaving the liquid beta-radiation emitting in the catheter for a pre-determined period of time, for example for less than 20 minutes, and preferably less than 15 minutes, depending on the dose necessary for the treatment and the specific activity of the source, (III) removing the liquid beta-radiation emitting from said catheter, and (IV) withdrawing the catheter. Preferably, the catheter is a balloon catheter. However, it can also be a canula or another suitable tubular device to accommodate a liquid beta-radiation emitting source.

In order to avoid a long ischemia time and distal embolizations and to be less cumbering for the patient a short irradiation time is desirable. However, the radiation exposure time depends on the vessel calibre, the morphology of the plaque, and the activity of the applied radiation emitting source [Chie E. K., Chae I. H., Lee M. M., Wu H. G. “Exposure of the operator to ionizing radiation during intracoronary radiation therapy.” J. Interv. Cardiol. 2002; 15(1): 15-18; Kim K. I., Bae J., Kang H. J., Koo B. K., Youn T. J., Kim S. H. Chae I. H., Kim H. S., Sohn D. W., Oh B. H., Lee M. M., Park Y. B., Choi Y. S., Lee D. S. “Three-year clinical follow-up results of intracoronary radiation therapy using a rhenium-188-diethylene-triamine-penta-acetic-acid-filled balloon system.” Circ J. 2004; 68(6): 532-537] and is determined before the treatment. In order to receive a sufficient dose within the vessel wall the inventors have established the following values for orientation: for an average minimal intima/media distance of 0.81 mm measured from the catheter surface the irradiation dose of 40 Gy should not be exceeded. For an average maximal distance of the media/adventitia boundary with a depth of 1.92 mm measured from the catheter surface at least 10 Gy irradiation dose should be received [Wolgemuth W. A., Leissner G., Kirchhoff K., Weissig T., Wengenmeaiser H., Sciuk J., Bohndorf K. “Intravaskuläre Ultraschallmessung von femoropoplitealen Plaques nach angiographisch erfolgreicher PTA—Bestimmung der Plaquemorphologie vor endovaskulärer Brachytherapie.” 88. Deutscher Röntgenkongress, Berlin, 18.5.2007, Abstract (submitted for publication)].

The liquid beta-radiation emitting source can be injected into the catheter for example by means of an applicator. The applicator can comprise a specific shield and a radioactive contamination free interface with the catheter in order to optimize the radiation protection, an integrated pressure control for the application in order to provide a simple and standardized application which considerably increases the security, and an integrated phiole in order to store the liquid beta-radiation emitting source for inflation and deflation. Preferably, the applicator is suitable to store Rhenium-188. The applicator can also include a means for rapid evacuation of the catheter in case a leak or rupture of the catheter is detected.

In another preferred embodiment of the method of a minimal invasive surgery according to the present invention the catheter of step b) is the balloon catheter of step a). In this case, the balloon catheter of step a) can be positioned in juxtaposition to the stenotic region and deflating the balloon catheter according to step (iii) is followed by injecting the liquid beta-radiation emitting source into the balloon catheter without changing the catheter. This embodiment has the advantage that no additional insertion of a catheter and additional preparatory steps like inserting a mandrel or a guide wire is necessary which causes additional inconvenience for the patient.

In another preferred embodiment of the method of a minimal invasive surgery according to the present invention the catheter of step b) is not the balloon catheter of step a). In this case the balloon catheter of step a) is withdrawn after deflating the balloon catheter according to step (iii) and a further catheter is inserted in juxtaposition of the stenotic region. In this embodiment the catheter used in step b) can be of the same type or of a different type than the catheter used in step a). For example, a catheter specifically designed for an irradiation of the stenotic region can be used. This catheter could have a larger or a smaller diameter than the catheter used in step a). It can be a balloon catheter or another type of catheter or a canula. In particular it can be a cardiologic catheter or a cardiologic catheter with overlength. Furthermore, a different material such as a material that is specifically suitable for a use of beta-radiation emitting sources can be used for the catheter.

The catheter used in step b) can be tested for leaks. Preferably, the catheter is placed under pressure with approximately 6 ml air using a Luer Lock syringe and in parallel, immersed in a sterile saline solution. Ascension of air bubbles in the saline solution would show an existing leakage. In this case the catheter has to be replaced.

In one embodiment of the method of a minimal invasive surgery according to the present invention the catheter of step b) is placed in juxtaposition to the stenotic region before step (I) according to the present invention. However, it is also possible to use a catheter that is already at least partially filled.

In another embodiment of the method of a minimal invasive surgery according to the present invention step (I) further comprises inflating the catheter by injection of the liquid beta-radiation radiation emitting source with a pressure between 0.5 to 7 bar, preferably between 2 to 5 bar, and more preferably of about 3 bar. The use of such low pressures compared to the pressure used for a dilatation of a stenotic region reduces the risk of a balloon rupture followed by an accidental incorporation of the liquid beta-radiation emitting source within step b). Inflating will automatically center the catheter within the vessel and further, will copy the individual morphology of the plaque of the patient. Therefore, a homogenous irradiation of the vessel wall will be achieved irrespective of irregular configured plaque or strong cambered vascular sections.

In yet another preferred embodiment of the method of a minimal invasive surgery according to the present invention the liquid beta-radiation emitting source has an activity in a range between 2500 and 30000 MBq/ml and preferably in a range between 3700 and 6600 MBq/ml. A high activity reduces the duration of the treatment which is favourable for the patient and also for the clinical personnel.

In a further preferred embodiment of the method of a minimal invasive surgery according to the present invention the liquid beta-radiation emitting source comprises Rhenium-188. However, also other sources can be used such as strontium-89, phosphor-32 or yttrium-90 or a combination of several sources.

Rhenium-188 shows a rapid dose decline within the tissue. In particular, Rhenium-188 shows a dose decline of a third of the dose in 1.1 mm distance from the surface of the catheter. Therefore, the vessel wall is selectively irradiated, wherein the surrounding tissue is preserved. Contrary to the use of gamma-sources no beta-radiation will leak from the human to be treated. Therefore, step b) can be performed in the same room as step a), preferably in an angiography unit, and the human to be treated has not to be transported in a specific radiation unit. In addition, it is now possible to monitor the endovascular brachytherapy and therefore to control the location of the radiation-emitting source in the body.

The Rhenium-188 is preferably eluted on the day of irradiation from a Wolfram-188 generator. Preferably, the eluted Rhenium-188 has an activity of at least 3.700 MBq/ml.

In one embodiment of the method of a minimal invasive surgery according to the present invention the method further comprises monitoring of restenose and vascular dissection. Monitoring of restenose and vascular dissection helps to evaluate the success of the endovascular angioplasty. The performing of an endovascular brachytherapy is reasonable only if a satisfactory result is achieved in step a).

In one preferred embodiment of the method of a minimal invasive surgery according to the present invention monitoring of less than 50% restenose and limited vascular dissection, preferably less than 30% restenose and no vascular dissection as a result of step a) leads to step b). In the context of the present invention “vascular dissection” means any hemodynamically relevant vascular dissections.

In one embodiment of the method of a minimal invasive surgery according to the present invention the vascular disease comprises arterial and venal vascular diseases.

In another preferred embodiment of the method of a minimal invasive surgery according to the present invention the arterial vascular disease comprises a peripheral arterial occlusive disease.

In one further preferred embodiment of the method of a minimal invasive surgery according to the present invention the peripheral arterial occlusive disease comprises a peripheral arterial occlusive disease stage IIb, III or IV according to Fontaine.

In yet another preferred embodiment of the method of a minimal invasive surgery according to the present invention said vascular disease is an in-stent stenosis.

In still another preferred embodiment of the method of a minimal invasive surgery according to the present invention said method is used in a cardiologic application.

The inventors have shown that one indication for an endovascular brachytherapy is the treatment of a human suffering from a peripheral arterial occlusive such as claudicatio intermittens (stage IIb according to Fontaine: walking distance less than 200 m) and critical ischemia, respectively (stage III according to Fontaine: pain while resting and stage IV according to Fontaine: necrosis and gangrene) [Wohlgemuth W. A., “Evidenzbasierte Einflussfaktoren und gesundheitsbezogene Lebensqualität—Eine gesundheitswissenschaftliche Analyse anhand der peripheren arteriellen Verschlusskrankheit. Habilitationsschrift”, Institut für Medizinmanagement und Gesundheitswissenschaften, Universität Bayreuth, Jan. 19, 2005]. Alone in Germany up to date an incidence of PTAs of approximately 64, 4 PTAs per 100.000 habitants per year is assumed [Wohlgemuth W. A., Freitag M. H., Wolfle K. D., Bohndorf K., Kirchhof K. “Incidence of major amputations, bypass procedures and percutaneous transluminal angioplasties (PTA) in the treatment of peripheral arterial occlusive disease in a German referral center 1996-2003” Rofo. 2006; 178(9): 906-910].

However, the indication further requires that the stenosis is capable to be treated percutaneously which means that the morphology and length of the stenosis allows the treatment using endovascular therapy. In addition, since endovascular brachytherapy is usually followed by administering anticoagulants, the human to be treated must not have any contradiction against these drugs.

In one embodiment of the method of a minimal invasive surgery according to the present invention the method further comprises step c) administering of perchlorate, preferably of natrium perchlorate. However, step c) is only required in case a balloon leakage and/or rupture occurs which is typically followed by an accidental of Rhenium-188.

### EXAMPLE

The present invention is described in the following by means of an exemplifying embodiment.

A patient suffering from an arterial or a venal vascular disease that is to receive a endovascular angioplasty of a stenotic region and a subsequent endovascular balloon brachytherapy at the dilated stenotic region is placed in a surgery room which preferably includes an angiography unit. The physician introduces a balloon catheter over a guidewire into the endovascular system and directs it to the stenotic region to which it is placed in juxtaposition. Subsequently, the balloon catheter is inflated to a pressure of 10 bar by injecting a sterile saline solution in order to achieve a dilatation of the stenotic region. Thereafter, the balloon catheter is deflated and removed from the patient.

The result of the endovascular angioplasty is monitored directly at the angiographic table in order to evaluate its success. In case of a positive result a second catheter such as balloon catheter which can be specifically designed for a brachytherapy using a beta-radiation source is inserted over a guide wire in the stenotic region that was previously reopened or dilated.

A beta-radiation emitting liquid including Rhenium-188 is eluted from a Rhenium-188 generator and is injected into the catheter with a pressure of 2 bar. The Rhenium-188 containing liquid has an activity of 3.700 MBq/ml. It remains in the catheter for a period of 15 minutes. The period of time was determined according to the measured activity of the beta-radiation emitting liquid and the medical prescription. Subsequently, the beta-radiation emitting liquid is evacuated from the catheter and the catheter is removed.

Thereafter, the catheter that was used for the balloon brachytherapy and the liquid containing Rhenium-188 are disposed. Possibly, the catheter and the liquid containing Rhenium-188 are recycled and sterilized for a further use.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A method of a minimal invasive surgery for the treatment of a human suffering from a vascular disease comprising the steps of:
a) performing an endovascular angioplasty of a stenotic region, and
b) performing an endovascular brachytherapy at said stenotic region using a liquid beta-radiation emitting source.

2. The method according to claim 1, wherein step a) comprises the steps of:
(i) placing a balloon catheter in juxtaposition to said stenotic region,
(ii) inflating said balloon catheter, and
(iii) deflating said balloon catheter.

3. The method according to claim 2, wherein step (ii) is performed with a pressure between 8 to 17 bar.

4. The method according to claim 2, wherein step (ii) is performed with a pressure between 10 to 15 bar.

5. The method according to claim 2, wherein step a) further comprises implanting a stent in juxtaposition to said stenotic region.

6. The method according to claim 2, wherein step a) further comprises withdrawing said balloon catheter.

7. The method according to claim 1, wherein step b) comprises the steps of:
I) injecting said liquid beta-radiation emitting source into a catheter,
II) leaving said liquid beta-radiation emitting in said catheter for less than 20 minutes,
III) removing said liquid beta-radiation emitting from said catheter, and
IV) withdrawing said catheter.

8. The method according to claim 1, wherein step b) comprises the steps of:
I) injecting said liquid beta-radiation emitting source into a catheter,
II) leaving said liquid beta-radiation emitting in said catheter for less than 15 minutes,
III) removing said liquid beta-radiation emitting from said catheter, and
IV) withdrawing said catheter.

9. The method according to claim 7, wherein said catheter is the balloon catheter of step a).

10. The method according to claim 7, wherein said catheter is not the balloon catheter of step a).

11. The method according to claim 10, wherein said catheter is placed in juxtaposition to said stenotic region before step (I).

12. The method according to claim 7, wherein step (I) further comprises inflating said catheter by injecting said liquid beta-radiation radiation emitting source with a pressure between 0.5 to 7 bar.

13. The method according to claim 7, wherein step (I) further comprises inflating said catheter by injecting said liquid beta-radiation radiation emitting source with a pressure between 2 to 5 bar.

14. The method according to claim 7, wherein step (I) farther comprises inflating said catheter by injecting said liquid beta-radiation radiation emitting source with a pressure of about 3 bar.

15. The method according to claim 12, wherein said liquid beta-radiation emitting source has an activity in a range between 2,500 and 30,000 MBq/ml.

16. The method according to claim 12, wherein said liquid beta-radiation emitting source has an activity in a range between 3,700 and 6,600 MBq/ml.

17. The method according to claim 15, wherein said liquid beta-radiation emitting source comprises Rhenium-188, strontium-89, phosphor-32, or yttrium-90 or any combination thereof.

18. The method according to claim 1, further comprising monitoring restenose and vascular dissection.

19. The method according to claim 18, wherein monitoring of less than 50% restenose and limited vascular dissection as a result of step a) leads to step b).

20. The method according to claim 18, wherein monitoring of less than 30% restenose and no vascular dissection as a result of step a) leads to step b).

21. The method according claim 1, wherein said vascular disease comprises arterial and venal vascular diseases.

22. The method according to claim 21, wherein said arterial vascular diseases comprises peripheral arterial occlusive diseases.

23. The method according to claim 22, wherein said peripheral arterial occlusive diseases comprise peripheral arterial occlusive diseases stage IIb or III according to Fontaine.

24. The method according claim 1, wherein said vascular disease is an in-stent stenosis.

25. The method according claim 1, wherein said method is used in a cardiologic application.
